(19) United States
(12) Patent Application Publication
(10) Pub. No.: US 2009/0093648 A1
(43) Pub. Date: Apr. 9, 2009

(54) Soapstock treatment

(76) Inventors: Ian C. Purtle, Plymouth, MN (US); Ahraon M. Eyal, Jerusalem (IL); Asher Vitner, Jerusalem (IL)

Correspondence Address: CARGILL, INCORPORATED, LAW/24, 15407 MCGINTY ROAD WEST, WAYZATA, MN, 55391, US

(21) Appl. No.: 11/547,545

(22) PCT Filed: Mar. 29, 2005
(86) PCT No.: PCT/US2005/010480

§ 371 Date: Nov. 14, 2008

Related U.S. Application Data
(60) Provisional application No. 60/557,200, filed on Mar. 29, 2004.

(51) Int. Cl. C07C 51/42; C11B 3/00

(52) U.S. Cl. 554/124; 554/205

(57) ABSTRACT

A method for the recovery of fatty acids comprising a salt of the fatty acid is disclosed. The method comprises the steps of: (a) reacting the salt of the fatty acid with CO₂ and with a reagent other than hydroxide and selected from a group of compounds carrying at least one of O—H, N—H, S—H, C—O—C and C—O—N moieties, to form a reaction mixture comprising at least one of a carbonate and a bicarbonate and a product selected from fatty acids and derivatives thereof, and (b) separating the product from the reaction mixture. A method for the production of fatty acid ester from free fatty acid of crude vegetable oil is also disclosed. A free fatty acid that is substantially free of emulsifier is also disclosed.

### CROSS-REFERENCES TO RELATED APPLICATIONS

The following patent applications are cross-referenced and are hereby incorporated by reference in their entirety: U.S. Patent Application No. 60/557,200 entitled “SOAPSTOCK TREATMENT SYSTEM” filed Mar. 29, 2004 as Attorney Docket No. CGL04/0011 USP1; PCT Patent Application No. US05/004200, entitled “EFFLUENET TREATMENT” filed Feb. 9, 2005 as Attorney Docket No. CGL03/0349WO1; PCT Patent Application No. US05/004160 entitled “PHENOLIC COMPOUND PURIFICATION” filed Feb. 9, 2005 as Attorney Docket No. CGL04/0008/WO1; PCT Patent Application No. US05/04153 entitled “PHENOLIC COMPOUND PURIFICATION” filed Feb. 9, 2005 as Attorney Docket No. CGL04/0009WO01; U.S. Patent Application No. 60/630,137 entitled “MONOSACCHARIDE PRODUCTION SYSTEM” filed Nov. 22, 2004 as Attorney Docket No. CGL04/0135USP1; PCT Patent Application No. 60/543,039 entitled “CYCLITOL SEPARATION METHOD” filed Feb. 9, 2005 as Attorney Docket No. CGL03/0489USP1; U.S. Patent Application No. 60/557,181 entitled “ISOFLAVONE DISTRIBUTION SYSTEM” filed Mar. 29, 2004 as Attorney Docket No. CGL04/0049USP2; U.S. Patent Application No. 60/557,199 entitled “ISOFLAVONE DISTRIBUTION SYSTEM” filed Mar. 29, 2004 as Attorney Docket No. CGL04/0049USP1; U.S. Patent Application No. 60/557,204 entitled “PROTEIN PURIFICATION SYSTEM” filed Mar. 29, 2004 as Attorney Docket No. CGL04/0093USP1.

### FIELD OF THE INVENTION

The present invention generally relates to a soapstock treatment. The present invention more particularly relates to a soapstock treatment and fatty acid production system and method. The present invention more particularly relates to a system and method for converting fatty acid salts of soapstock to free fatty acid or to fatty acid derivatives, such as esters. The present invention more particularly relates to a treatment system where free fatty acids or its esters are recovered from soapstock and where the recovery is done with substantially no consumption of a mineral acid.

### BACKGROUND OF THE INVENTION

Extracted vegetable oils are mainly composed of triglycerides in which three fatty acid molecules are esterified to a glycerol molecule. Extracted crude oil also contains impurities such as phospholipids and free fatty acids. Such impurities are typically removed from crude oil in a process typically referred to as “vegetable oil refining.” Such vegetable oil refining typically involves degumming, i.e. contacting with water followed by the removal of hydrated phospholipids (gums), e.g. through centrifugation. Typically, vegetable oil refining also Involves alkali treatment, i.e. removal of free fatty acids by contacting the vegetable oil with an alkaline solution. The free fatty acids (RCOOH) react with the alkali to form their alkaline salt (soap), as in reaction (i), where the alkali may be sodium hydroxide.

RCOOH+NaOH→RCOONa+H2O (i)

The formed salts are separated out, e.g. by centrifugation, as a soapstock. The soapstock typically contains entrained vegetable oil, water, and non-hydratable phosphatides (NHP)—and in many cases some unreacted alkali. According to an alternative known method, gums removal and alkali-treatment are conducted simultaneously or sequentially without pre-removal of gums, so that the co-product separated, e.g. by centrifugation, contains gums, fatty-acid salts and entrained vegetable oil. This separated co-product is also referred to herein as soapstock. Soapstock is generated by the industry in large amounts, and may be used as a component in animal feed.

Fatty acids are of commercial value for use as such and/or as reagents for other products, e.g. fatty acid methyl esters used in biodiesel. A conventional acidulation process for the recovery of free fatty acids from soapstock is known. According to such known acidulation process, a reaction with a strong mineral acid liberates the fatty acids from the salts to form a free fatty acid and a salt of the mineral acid, as in reaction (ii) for the case where the mineral acid is sulfuric.

2RCOONa+H₂SO₄→2RCOOH+Na₂SO₄ (ii)

According to such known acidulation process, the reaction products are separated, e.g. by centrifugation. Presence of emulsifiers, such as gums and NHP interferes with phase separation. Three phases are formed: an organic phase containing the free fatty acids, an aqueous phase comprising the mineral acid salt and other solutes, and a sludge phase.

Such known acidulation process enables recovery of free fatty acids. However, such known acidulation process has several disadvantages including reagent consumption and byproduct salt disposal as demonstrated by the reagents and products of the overall process:

$$\frac{\begin{matrix}2 R COOH + 2 NaOH \to 2 R COONa + 2 {H}_{2} O \\ 2 R COONa + {H}_{2} {SO}_{4} \to 2 R COOH + {Na}_{2} {SO}_{4}\end{matrix}}{ 2 NaOH + {H}_{2} {SO}_{4} \to 2 {Na}_{2} {SO}_{4} + 2 {H}_{2} O} \qquad \begin{matrix}\begin{matrix} ( i ) \\ ( ii ) \end{matrix} \\ ( iii ) \end{matrix}$$

As shown in reactions (i) through (iii), fatty acids start as free fatty acids (in the crude vegetable oil) and end up in the same form after acidulation. Fatty acid salts are formed in the first reaction and decomposed in the second. The only overall chemical change is the introduction of a base and of a mineral acid and the formation of a byproduct salt. Thus, the separation of fatty acids from vegetable oil according to the scheme above consumes an alkali and an acid as reagents and forms a byproduct salt. In addition, there are costs related to the disposal of the aqueous phase comprising that salt and other solutes.

Accordingly, there is a need for a soapstock treatment and fatty acid production system that does not necessarily require the consumption of a mineral acid and the production of a mineral salt. There is also a need for a soapstock treatment and fatty acid production system that, when combined with alkali-treatment of crude vegetable oil regenerates the alkali to be reused in the treatment, obviating or minimizing thereby alkali consumption. It would be advantageous to provide a soapstock treatment filling any one or more of these needs or having other advantageous features.

### SUMMARY OF THE INVENTION

The present invention relates to a method for the recovery of fatty acids comprising a salt of the fatty acid. The method comprises the steps of: (a) reacting the salt of the fatty acid with CO₂ and with a reagent other than hydroxide and selected from a group of compounds carrying at least one of O—H, N—H, S—H, C—O—C and C—O—N moieties, to form a reaction mixture comprising at least one of a carbonate and a bicarbonate and a product selected from fatty acids and derivatives thereof, and (b) separating the product from the reaction mixture.

The present invention also relates to a method for the production of fatty acid ester from free fatty acid of crude vegetable oil. The method includes: (a) treating the crude vegetable oil with at least one of bicarbonate and carbonate to form a salt of the fatty acid; (b) separating the salt of the fatty acid from the crude vegetable oil to form a fatty-acid-depleted vegetable oil and a fatty-acid-salt-containing soapstock; (c) reacting the salt of the fatty acid with CO₂ and an alkanol to form a reaction mixture comprising a fatty acid ester and at least one of bicarbonate and carbonate of the cation of the salt; (d) separating the ester from the reaction mixture; (e) separating the at least one bicarbonate and the carbonate from the reaction mixture; and (f) using at least a fraction of the separated at least one of bicarbonate and carbonate in the treatment of step (a).

The present invention also relates to a free fatty acid that is substantially free of emulsifier. The free fatty acid is produced by the process of: (a) reacting a salt of the fatty acid with CO₂ and with a reagent other than hydroxide and selected from a group of compounds carrying at least one of O—H, N—H, S—H, C—O—C and C—O—N moieties, to form a reaction mixture comprising at least one of a carbonate and a bicarbonate and the free fatty acid, and (b) separating the free fatty acid from the reaction mixture.

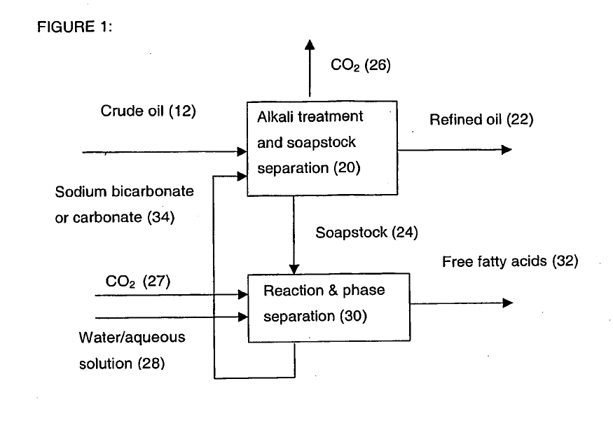

FIG. 1 is a flow diagram of alkali treatment of crude oil with recycled sodium bicarbonate followed by treatment of the formed soapstock to form free fatty acids and bicarbonate to be recycled to alkali treatment according to an exemplary embodiment of the present invention.

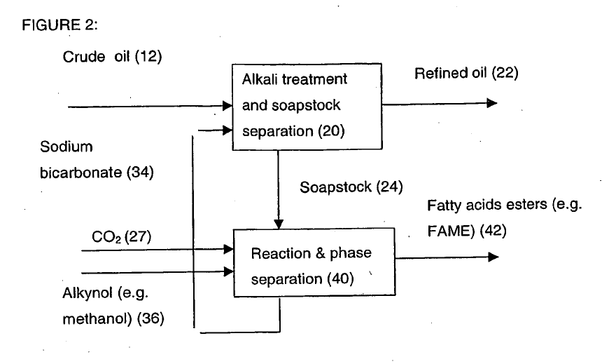

FIG. 2 is a flow diagram of alkali treatment of crude oil with recycled sodium bicarbonate followed by treatment of the formed soapstock to form fatty acid methyl ester and bicarbonate to be recycled to alkali treatment according to an alternative embodiment of the present invention.

### DETAILED DESCRIPTION OF THE PREFERRED AND OTHER EXEMPLARY EMBODIMENTS

FIG. 1 shows an exemplary embodiment of a treatment system for the recovery of a fatty acid from a soapstock, which may be integrated with alkali treatment of crude oil according to an alternative embodiment. Referring to FIG. 1, a soapstock comprising sodium salts of fatty acids (24) may be formed in an alkali treatment of crude vegetable oil (20). In the exemplary embodiment of FIG. 1, crude vegetable oil (12) is reacted with sodium bicarbonate or carbonate from a previous step (34) to convert free fatty acids in the crude oil to their sodium salt. The reaction is followed with a separation step, forming refined oil with reduced fatty acid content (22) and the soapstock (24) that contains the sodium salt of the free acid. The soapstock may be reacted with CO₂ (26) and water (28, and sodium bicarbonate and free fatty acid are formed in the reaction. The fatty acid may be separated from the reaction mixture to form a free fatty acid product (32) and used as such or further treated. According to a preferred embodiment, the sodium bicarbonate is separated (28) and reused (e.g. recycled) in alkali refining of crude oil. The treatment regenerates an alkali for crude oil treatment.

According to a preferred embodiment, the reaction in (30) is conducted in a pressure vessel. According to another preferred embodiment, the partial vapor pressure of CO₂ may be greater than about 1 Kg/m² during at least a portion of the duration of the reaction.

According to a particularly preferred embodiment, a mole of water is consumed in the reaction per mole of fatty acid salt converted to free fatty acid. According to a preferred embodiment, more water is present than the stoichiometric requirement. According to a preferred embodiment, water to fatty acid salt weight ratio is in the range of between about 0.1 and about 100, preferably between about 2.5 and about 40, more preferably about 3 and about 10. Water may be added (28) as such or as an aqueous solution according to alternative embodiment. According to another alternative embodiment, the added water may be from an oil wash operation.

In the presence of water, the preferred reaction temperature could be in the range between about 0 C. and about 95 C, more preferably between about 20 C and about 70 C, most preferably between about 30 C and about 50 C.

The soapstock may contain phospholipids, phospholipid derivatives, such as lyso-phospholipids and/or non-hydratable phosphatides (NHP). According to a preferred embodiment, the NHPs are hydrolyzed. Hydrolysis is conducted before the reaction with CO₂, according to a preferred embodiment. Fatty acids formed as a result of that hydrolysis may be added to the free fatty acids formed. According to a preferred embodiment, such hydrolysis is conducted at an elevated temperature, e.g. greater than about 100 C and in a pressure vessel. If desired, alkali may be added to that hydrolysis step, e.g. part of the bicarbonate produced in the process according to an alternative embodiment. At about the end of the hydrolysis, the temperature of the soapstock may be adjusted to the one optimal for the reaction with CO₂. If entrained oil is present in the soapstock, such hydrolysis step may lead to hydrolysis of the oil, adding thereby more fatty acid to the soapstock and increasing thereby the fatty acid yield of the process according to an alternative embodiment.

Such hydrolysis of phospholipids, derivatives and/or NHP may minimize the interference of those in the separation of the formed free fatty acids from the reaction mixture. In cases where such separation presents no difficulty (e.g. due to low content of phospholipids and NHP in the soapstock or due to the selected separation method), the hydrolysis steps may be avoided.

According to a preferred embodiment as shown in FIG. 1, where water is used as a reagent and possibly also as a solvent, free fatty acids are formed. In case of full consumption of the fatty acid salts and in case the water content may be high enough to fully dissolve the formed bicarbonate, at the end of reaction there are three phases: (1) a CO₂-containing vapor phase; (2) a bicarbonate-containing aqueous solution; and (3) a free fatty acid-containing organic phase. In case of oil presence in the soapstock, and if that oil is not hydrolyzed, it ends up in the organic phase, according to a preferred embodiment. If the concentration of phospholipids and NHP is small (e.g. due to a separate degumming step or due to hydrolysis), phase separation (e.g. decantation or centrifugation) may be used to separate both products of the reaction. According to an alternative embodiment, an organic solvent is used for extracting the free fatty acid forming a free fatty acid-containing extract. If desired, free fatty acid can be separated from the extract, e.g. by distillation. According to any preferred or alternative embodiments, separation may be done either under CO₂ pressure or rapidly, in order to substantially avoid reversion of the reaction.

The treated soapstock may contain significant amounts of entrained oil, which represent oil losses in known industrial practice. According to an embodiment of the soapstock treatment method, those oil values are recovered. According to an embodiment, the entrained oil is hydrolyzed to form free fatty acids or their salt, which are separated along with the crude oil fatty acids. According to an alternative embodiment, the entrained oil is not hydrolyzed and separated as such in the method of soapstock treatment. In cases where the free fatty acid formed in the reaction are separated by phase separation, the entrained oil could be kept in the same phase as the free fatty acids and then separated from those, e.g. by distillation of the free fatty acid. In case of separating free fatty acid by solvent extraction, the entrained oil could be co-extracted with the free fatty acid and then separated from those before, after or simultaneously with solvent removal. The separated oil is combined with crude oil, e.g. degummed and alkali-treated oil, according to a preferred embodiment or at another point in the process of separately treated according to alternative embodiments.

According to an alternative embodiment, the reaction may be not pushed to completion so that fatty acid salts are present in the reaction mixture. In such cases, the products are separated from the fatty acid salts and the latter are recycled to the reaction. Separation of other components of the reaction mixture, e.g. entrained oil, is combined with the product-salt separation, according to a preferred embodiment. Such mode of operation enables formation of bicarbonate solutions of higher concentration. In those case, the vapor phase and the aqueous phase are preferably removed first, leaving an organic phase comprising a mixture of free fatty acids, fatty acid salts, and optionally also entrained oil, all of which having low water solubility. In the absence of the bicarbonate, which is already separated with the aqueous phase, there may be substantially no reversion of the reaction. Several methods are suitable for separation of the free acids from the salts, including gravimetric separation, phase separation, distillation, solvent extraction, etc.

For phase separation the mixture temperature is adjusted to where the free fatty acids are in a liquid form, while the salts are in a solid form according to a particularly preferred embodiment. Phase separation therefore forms a liquid phase rich in free fatty acids (and oil, if present) and a solid phase rich in fatty acid salts. Preferred temperatures are selected based on the melting points of the fatty acid, typically in the range between 30 and 80 C. The melting points of the salts are substantially higher. In cases where oil is also present, temperature is selected so that the oil is also in liquid form, according to a preferred embodiment.

In case of separating via solvent extraction, according to an alternative embodiment, an extractant may be mixed with the acids/salts mixture. The free fatty acids dissolve into the extractant, which may be separated by phase separation. Many solvents are suitable, e.g. hexanes, other hydrocarbons, alkanols, esters, etc. according to any preferred or alternative embodiments. The free fatty acids may be recovered from the extractant solution, e.g. by distillation of the extractant. The extractant may be selected according to local preference, e.g. using hexanes of oil extraction.

The solvent may be oil, preferably crude oil before alkali treatment. According to a preferred embodiment, the crude oil used as a solvent is a relatively small fraction taken from the oil before its introduction to the alkali treatment that then generates the soapstock to be treated. According to a preferred embodiment, crude oil is already present in the reacting step. Oil present in the reaction step may be the oil entrained in the soapstock, added crude oil, or both according to alternative embodiments. In treatment systems where both water and oil are present in the reaction step, the water to oil weight ratio may be in a range of between about 1 and about 20, preferably between about 2 and about 5.

According to an alternative embodiment, when the liberated fatty acids are further processed, the extractant may be selected based on the requirements of further processing. For example, the fatty acids are reacted with alkanols to form the corresponding esters for various applications, e.g. to methyl esters for use as bio-diesel. In those cases, the selected alkanol may be used as the extractant or a component thereof.

In case of product separation by phase separation or by extraction, oil originally entrained in the soapstock follows the free fatty acid and may be separated from those, e.g. by distillation of the acid. In case of distilling the fatty acids out of the reaction mixture, the oil stays with the fatty acid salts and may be separated from those e.g. by phase separation or solvent extraction. In case of product separation by solvent extraction, the oil follows the free fatty acid and could be separated, e.g. by distillation of the acid or by temperature adjustment.

The bicarbonate formed may be either in crystalline form or in an aqueous solution. According to a preferred embodiment, it may be re-used in alkali-treatment of crude oil, as shown in stream (34) of FIG. 1. According to an alternative embodiment, pre-treatment may be conducted before the re-use. Such pre-treatment may involve concentration of the solution, e.g. by water evaporation or reverse osmosis, removal of impurities, e.g. by adsorption, crystallization or bleeding, etc.

According to an alternative embodiment, e.g. for increasing basicity, the bicarbonate solution may be heat treated, whereby the bicarbonate is converted to carbonate (and CO₂ is liberated). Thus, according to that embodiment, carbonate may be used in the alkali treatment, bicarbonate may be formed in the soapstock reaction with CO₂ and converted to bicarbonate by heat treatment before recycle to the alkali treatment. According to an alternative embodiment, the bicarbonate/carbonate mixture is formed in the reaction with CO₂.

Since the alkali may be kept in a closed cycle, there may be build up of impurities, particularly water-soluble ones. Those may be removed by treating the recycled stream, as explained above. According to an alternative embodiment, a bleeding operation may be introduced, wherein impurities are purged out from the recycled stream. According to an alternative embodiment, impurities are purged out of the soapstock before the reaction with CO₂.

Referring further to FIG. 1, sodium bicarbonate may be used in the alkali treatment and may be regenerated in the reaction with CO₂. Other carbonates or bicarbonates are also suitable, e.g. those of other alkali and alkaline earth metals and of ammonia according to alternative embodiments. According to alternative embodiments, relatively higher cost bases, such as potassium carbonate or bicarbonate, may be selected, since substantially all the base is reformed and reused. In that case, the soapstock comprises potassium salts of the fatty acids.

The process may be conducted in a batch or in a continuous way according to any preferred or alternative embodiments. According to a preferred embodiment, water is present in the reaction and the reaction is conducted in a multiple-stage counter-current mode of operation. In that case, on one side water or an aqueous solution may be fed and free fatty acids exit, while on the other, the soapstock may be entered and a solution of bicarbonate exits.

Alkali treatment of fatty-acid-comprising crude oil with carbonate or bicarbonate liberates CO₂ ((26) in FIG. 1). CO₂ is also liberated in those cases where the reaction with CO₂ generates a bicarbonate, which is converted to carbonate prior to reuse in alkali treatment of crude oil. The liberated CO₂ in either of those process, or in both, is collected, according to a preferred embodiment and reused in the reaction, forming a significant fraction, or practically all the CO₂ fed to the reaction ((27) in FIG. 1). It required, the collected CO₂is compressed prior to such reuse.

FIG. 2 shows an alternative embodiment of a treatment system for the recovery of a fatty acid f rom a soapstock, which may be integrated with alkali treatment of crude oil. Referring to FIG. 2, a soapstock comprising sodium salts of fatty acids (24) is formed in an alkali treatment of crude oil as in the embodiment of FIG. 1. The soapstock is reacted in (40) with CO₂ (27) and with an alkanol, such as methanol (36). Sodium bicarbonate and methyl esters of the fatty acids are formed in the reaction. The ester is separated from the reaction mixture to form an ester containing stream (42), which can be used is as such or further treated. In cases where the alkanol is methanol, the product ester is fatty acid methyl ester (FAME), suitable for use in biodiesel. According to a preferred embodiment, the sodium bicarbonate is separated to form an alkali stream (34) and reused in alkali refining of crude oil. As in the case of the embodiment shown in FIG. 1, the overall process may obviate the consumption of a mineral acid and of an alkali and the production of mineral salt to be disposed off, through regenerating an alkali for crude oil treatment. Use of alkanols other than methanol results in the formation of the corresponding esters. Such alkanols include glycerol, forming mono-, di- or triglycerides in the reaction. Poly-hydric alkanols may also be used in their partially esterified form.

Alternatively to alkanols, other reagents that react with the acid function of fatty acid could be used, e.g. esters, amines (with at least one N—H moiety), amides, etc could be used. The produced fatty acid product changes according to the reagent.

The reagent, e.g. alkanol, may be used in amounts larger than stoichiometric and serve as a solvent according to alternative embodiments.

In case of phospholipids presence in the soapstock, the alkanol used may react with those too to form esters of fatty acid originally bound in the phospholipids according to an alternative embodiment.

The fatty acid products, e.g. esters are separated from the reaction mixture by methods similar to those for separation of free fatty acids according to any preferred or alternative embodiment.

The reaction with CO₂ and alkanol may be optionally conducted in the presence of a suitable catalyst, e.g. sodium methoxide or organic compounds of transition metals. According to an alternative embodiment, enzymatic catalysis, e.g. by lipase, may be used.

### EXAMPLES

While the invention will now be described in connection with certain embodiments in the following examples so that aspects thereof may be more fully understood and appreciated, the examples are not intended to limit the invention to these particular examples.

### Example 1

Sodium stearate and water at weight ratio of 15:85, were introduced into a pressure vessel, the temperature was adjusted to 90 C and CO₂ was introduced. CO₂ pressure was maintained at 30 atmospheres and mixing was applied. After 2 hours, mixing was stopped and the phases were separated. The organic phase was analyzed for free fatty acid and the aqueous phase for sodium bicarbonate. The analyses showed 30% conversion of the sodium stearate to free stearic acid.

### Example 2

Example 1 was repeated at similar conditions, but the reaction time was doubled. The conversion yield was about 50%.

### Example 3

Example 1 was repeated with the following changes: the water proportion was increased to 97.5% of the starting mixture, the temperature was reduced to 68 C, the pressure was increased to 39 atmospheres and the reaction duration was 5 hours. The analyses at the end of the reaction showed conversion yield greater than 90%.

### Example 4

Example 1 was repeated with the following changes: The reaction system contained 5% of the salt, 5% of water and 90% methanol, reaction temperature was 135 C, CO₂ pressure was 20 atmospheres and the reaction duration was 16 hours. The analyses at the end of the reaction showed that 10% of the fatty acid in the salt was converted into their methyl ester.

While the preferred and other exemplary embodiments described in this disclosure are presently preferred, it should be understood that these embodiments are offered by way of example only. For example, the process does not necessarily require the presence of a water-immiscible base such as amine or an anion exchanger. The invention is not limited to a particular embodiment, but extends to various modifications, combinations, and permutations.

What is claimed is:

1. A method for the recovery of fatty acids comprising a salt of the fatty acid, the method comprising the steps of: (a) reacting the salt of the fatty acid with CO₂ and with a reagent other than hydroxide and selected from a group of compounds carrying at least one of O—H, N—H, S—H, C—O—C and C—O—N moieties, to form a reaction mixture comprising at least one of a carbonate and a bicarbonate and a product selected from fatty acids and derivatives thereof, and (b) separating the product from the reaction mixture.

2. The method of claim 1, wherein the reagent is selected from a group consisting of water, alkanols, esters and combinations thereof.

3. The method of claim 1, wherein the product comprises at least one of free fatty acids and fatty acid esters.

4. The method of claim 1, wherein a cation of the salt of the fatty acid is selected from a group of cations of ammonia, alkali and alkaline earth metals and combinations thereof.

5. The method of claim 1, wherein the salt of the fatty acid comprises a product of a vegetable oil refining process.

6. The method of claim 1, wherein the salt of the fatty acid comprises a soapstock of a vegetable oil refining process.

7. The method of claim 6, wherein the vegetable oil refining process is selected from a group consisting soybean oil, rapeseed oil, sunflower oil and combinations thereof.

8. The method of claim 1, wherein a partial vapor pressure of the CO₂ is greater than about 1 Kg/m² during at least a portion of duration of the reaction.

9. The method of claim 1, wherein the reaction with CO₂ is conducted substantially in the absence of a water-insoluble base.

10. The method of claim 1, wherein the reaction with CO₂ is conducted in the presence of at least one of water, vegetable oil and organic solvents.

11. The method of claim 10, wherein the reaction with CO₂ is conducted in the presence of water.

12. The method of claim 11, wherein the water to salt weight ratio is in a range of between about 0.1 and about 100.

13. The method of claim 10, wherein both water and vegetable oil are present and wherein the water to oil weight ratio is in a range of between about 1 and about 20.

14. The method of claim 11, wherein the reaction with CO₂ is conducted at a temperature of between about 0 C and about 95 C.

15. The method of claim 11, wherein the reaction with CO₂ is conducted in a multiple-stage counter-current mode, which is fed at least one of water and an aqueous solution on one side and the salt of the fatty acid on the other.

16. The method of claim 11, wherein the alt of the fatty acid is a product of vegetable oil refining, wherein such refining comprises a step of washing with water or with an aqueous solution an alkali-treated oil, and wherein the water present in the reaction results, at least partially, from wash of alkali-treated oil.

17. The method of claim 11, wherein the product is a free fatty acid.

18. The method of claim 1, wherein the separation of the product comprises at least one of solvent extraction, distillation, and gravimetric separation.

19. The method of claim 1, wherein the separation of the product uses solvent extraction and the solvent used is a vegetable oil.

20. The method of claim 1, further comprising the step of separating by distillation the product from a solvent comprising the vegetable oil.

21. The method of claim 1, wherein the product is a free fatty acid and wherein a fatty acid solution in the alkanol is formed.

22. The method of claim 21, wherein the fatty acid in the alkanol solution is reacted to form the ester of the fatty acid with the alkanol.

23. The method of claim 1, further comprising the step of separating at least one of the formed carbonate and the bicarbonate to form at least one of carbonate and bicarbonate product.

24. The method of claim 23, wherein at least one of the formed carbonate and bicarbonate product is used for alkali treatment of crude vegetable oil.

25. The method of claim 24, wherein the bicarbonate product is converted to carbonate product prior to the use in alkali treatment of crude vegetable oil.

26. The method of claim 24, wherein the at least one of the formed carbonate and bicarbonate product is an aqueous solution, further comprising a step of concentrating the aqueous solution by methods comprising at least one of reverse osmosis, water evaporation, and combinations thereof.

27. The method of claim 24, further comprising a step of bleeding an aqueous solution in order to avoid substantial build up of impurities.

28. The method of claim 27, wherein bleeding and reacting with CO₂ are conducted substantially simultaneously.

29. The method of claim 1, wherein the feed stream contains at least one of phospholipid and its derivatives and wherein at least one of phospholipid and the derivatives is hydrolyzed before the reaction with CO₂ or simultaneously with it.

30. The method of claim 1, wherein the reagent is an alkanol and the product is an ester of the alkanol.

31. The method of claim 1, wherein the reagent is methanol and the reaction product is a fatty acid methyl ester.

32. The method of claim 24 or 25, wherein CO₂ formed is collected and used in the reaction of step (a).

33. The method of claim 32, wherein the collected CO₂ is compressed prior to the use in the reaction of step (a).

34. The method of claim 1, wherein reacting with CO₂ does not reach completion so that the reaction mixture comprises unreacted fatty acid salts.

35. The method of claim 34, further comprising a step of separating unreacted fatty acid salts.

36. The method of claim 35, wherein at least a fraction of the separated unreacted fatty acid salts is recycled to the reaction.

37. The method of claim 1, wherein the salt of the fatty acid comprises vegetable oil, and further comprising a step of separating the vegetable oil from the reaction mixture to form a separated vegetable oil stream.

38. The method of claim 37, wherein the separated vegetable oil stream is combined with crude vegetable oil as such or after pretreatment.

39. A method for the production of fatty acid ester from free fatty acid of crude vegetable oil, comprising the steps of: (a) treating the crude vegetable oil with at least one of bicarbonate and carbonate to form a salt of the fatty acid; (b) separating the salt of the fatty acid from the crude vegetable oil to form a fatty-acid-depleted vegetable oil and a fatty-acid-salt-containing soapstock; (c) reacting the salt of the fatty acid with CO₂ and an alkanol to form a reaction mixture comprising a fatty acid ester and at least one of bicarbonate and carbonate of the cation of the salt; (d) separating the ester from the reaction mixture; (e) separating the at least one bicarbonate and the carbonate from the reaction mixture; and (f) using at least a fraction of the separated at least one of bicarbonate and carbonate in the treatment of step (a).

40. A free fatty acid that is substantially free of emulsifier, produced by the process of: (a) reacting a salt of the fatty acid with CO₂ and with a reagent other than hydroxide and selected from a group of compounds carrying at least one of O—H, N—H, S—H, C—O—C and C—O—N moieties, to form a reaction mixture comprising at least one of a carbonate and a bicarbonate and the free fatty acid, and (b) separating the free fatty acid from the reaction mixture.

41. The free fatty acid of claim 40 wherein the emulsifier is at least one of a gum and a non-hydratable phosphatide.
